(19) United States
(12) Patent Application Publication
(10) Pub. No.: US 2006/0251738 A1
(43) Pub. Date: Nov. 9, 2006

(54) Rock flour, especially dolomite-based medicament, for the treatment of cancer diseases

(76) Inventor: Gerd Thöne, Lage (DE)

Correspondence Address: MCCARTER & ENGLISH LLP;CITYPLACE I, 185 ASYLUM STREET, HARTFORD, CT, 06103, US

(21) Appl. No.: 10/558,099

(22) PCT Filed: May 18, 2004
(86) PCT No.: PCT/DE04/01047

§ 371 Date: Nov. 22, 2005

(30) Foreign Application Priority Data
May 22, 2003 (DE) 103237593

(51) Int. Cl. A61K 33/10; A61K 33/00

(52) U.S. Cl. 424/686; 424/724; 977/906

(57) ABSTRACT

The invention relates to a drug for internal application, especially for the treatment of cancerous conditions. The drug comprises minerals, for example, silica flour, dolomite flour, quartz, calcium, magnesium powder, or combinations thereof. In a preferred embodiment the drug of the invention includes rock flour minerals with a grain size of from about 1 nm to about 150 nm. In certain embodiments, the rock flour drug of the invention optionally comprises at least one other ingredient, for example, an oil, an excipient, a carrier, and the like.

The present invention relates to a drug for internal use, more specifically for treating cancerous conditions.

There has long been a need for a drug against cancerous conditions. Although drugs are known and utilized, they often have serious side effects. Also, the currently utilized drugs are very expensive, not least because they are complex to manufacture. Therefore, there is a continuous need for well-tolerated low-cost drugs in this field.

This is where the invention sets in. It is its object to indicate a drug for internal use, more specifically for treating cancerous conditions that permits to most effectively attack cancer tumors. The drug should have least side effects and be low in cost.

This object is solved by a drug having a fraction of minerals with a grain size in the range of 1 through 150, more specifically of 1 through 20 nanometers.

It is known from the European Patent EP 0 889 721, the disclosure of which is fully incorporated herein by reference, that silicon-based minerals such as quartz are beneficial to human well-being when used as a dietary supplement. This colloidal preparation, which is based on approximately 20 percent by weight of quartz flour that is ground to a colloidal solution together with ricinus oil, water, an emulsifier and other pulverized minerals, can be utilized to advantage in the external treatment of skin diseases. Only minerals in the colloidal and molecular grain size range are bioactive, whilst coarser grain sizes are not.

Further tests by the inventor showed that such finely ground minerals are also suited for the treatment of cancerous conditions.

The peculiarity of the invention is that the extremely finely distributed minerals have an infinitesimal diameter. They are distributed so finely that they will not remain on the surface of a tumor and be filtered away as a result thereof but that they are allowed to penetrate into the tumor. The diameter of the minerals is smaller than the diameter of a skin pore.

The medical tests performed permitted to find out that the drug has a particularly advantageous effect if the rock flour has fractions of quartz and dolomite. The effect was even further improved by adding magnesium oxide powder. Calcium has the synergetic effect of providing effective heartburn relief.

A just as effective drug is obtained if quartz flour is replaced by precipitated silicic acid. Thanks to its large absorbing surface, the colloidal distribution of silicon significantly facilitates tissue penetration and also the bioactive interaction between the human metabolism and the discrete silicon particles.

The effect of silicon in accordance with the invention is only obtained if it is present in the finely distributed bioactive form; that is to say in the form of nanoparticles. This is obtained by grounding the silicon acid previously subjected to chemophysical treatment to form a liquid substance in which the particles are not allowed to deposit so that the molecules cannot accumulate. This colloidal silicon is similar to the albinoid arrangement of crude proteins (without being sticky). It is similar to blood plasma. The colloidal compound results in a large absorption surface area (1 g silicon gel=300 m² absorption surface area) and allows for incorporation of the bioactive silicon into the blood and the connective tissue. Thanks to the large absorbing surface area, the colloidal distribution of the silicon significantly facilitates tissue penetration and the bioactive interaction between the human metabolism and the discrete silicon particles. The colloidal distribution and arrangement ensure external interfaces not only between liquid and air but also between silicon and the water molecules. Thus, silicon has a surface activity and internal stress energies that have a unique activating effect on the biological processes. It is suited both for internal and external use.

In this context, it should be noted that most naturally occurring silicon, even rock crystal, which consists of pure silicon, has no stimulating bioactive effect on the metabolism. As already mentioned, this property is only achieved by processing it into a colloidal preparation of nanoparticles.

Raw silicon is obtained from sand and coal and is further processed to the desired silicones in a continuous process. Natural gas or petroleum serves to produce methanol (synthesis gas), another starting material for the synthesis of silicones. Chlorine, which is supplied to the process in the form of HCl, is obtained by the electrolysis of rock salt solutions.

The drug of the invention may advantageously be complemented by the addition of water, oil (mineral oil or vegetable oil) or alcohol. With aqueous solutions, a fraction of about 60% by weight of water in the final product has been found to be suited. The oil used may advantageously be vegetable oil such as safflower oil which has no laxative effect when taken. An emulsion of polydimethylsiloxane and water is also possible.

The mineral drug of the invention is advantageously manufactured by mixing together the starting substances in a first step. It has thereby been found advantageous to have the grain size of rock flour used in the range of 10 micrometers or less. In a subsequent method step, the mixed components are ground using a colloid mill with the milling time being chosen to match the desired grain size of the final product. It has thereby been found advantageous to continue the milling process for at least 10 minutes, a rotor being indicated for cooling the colloid mill in order to prevent overheating.

The thus obtained mixture is a flowable gel-like liquid and can be processed into a drug in a variety of manners. It is for example possible to prepare a powder mixture by mixing the above liquid with zeolite and dolomite powder. This powder may then be supplied for example in the form of the widely used gelatine capsules that dissolve in the digestive tract. It is moreover readily possible to compress the powder mixture into tablets that may be taken with or without liquid.

It is also possible to inhale the drug of the invention. For inhalation, the drug is nebulized in an apparatus using appropriate nebulizing techniques. Diverse apparatus are available for this purpose:

1. Pneumatic Venturi nozzle nebulizer:
a) direct nebulization
b) with an aerosol reservoir
c) with an overpressurized solution to be inhaled.

2. Mechanical one-substance nozzle nebulizer

3. Ultrasonic nebulizing

4. Ultrasonic pressure through perforated screen

5. Ultrasound-operated perforated membrane

6. Electromechanical pressure through perforated membrane

Inhalation through an inhalation mask as well as with a mouthpiece or a nosepiece has been found to be well suited for this purpose.

The use of quartz flour, dolomite rock flour and magnesium powder has been found to be particularly advantageous. The fractions of zeolite and dolomite may add up to 100% by weight of the mass fraction of the rock flour.

In addition to the constituent substances mentioned, aluminium silicate, more specifically natural zeolite with a grain size in the range of 10 μm to 70 μm, more specifically of 40 μm, and dolomite powder with a grain size in the range of 2 μm to 30 μm, more specifically of 10 μm, may be added. A powder is thus obtained that may be administered in capsules.

The framework of the zeolite crystal lattice is mainly built from SiO₄ tetrahedrons. It comprises empty spaces containing ions such as sodium, potassium and calcium that can be readily interchanged and exchanged with their substrate environment. In living organisms, this mineral-specific crystal structure (cage structure) of zeolite has the excellent property of binding (absorbing) toxins such as ammonia and other nitrogen compounds but also heavy metals and to eliminate them through the digestive tract. The eliminated toxins are replaced by minerals the body urgently needs. Thus, the organism's homeostasis, more specifically the mineral metabolism, is maintained or re-established.

Beside the effect of healing cancerous conditions, another effect of the drug is that it not only protects against toxic damage delicate organ systems such as the brain, the nervous system, the hormonal system, the immune system, the liver, the kidneys and so on, but also increases their resistance to toxic pathogenic influences.

Like silicon, zeolite moreover has a positive stimulating influence upon the entire metabolism and on the growth and healing processes of the organism.

Thanks to its open molecule structure, zeolite is further capable of absorbing large amounts of liquid. This presents an advantage as it permits to form a flowable powder in spite of its being mixed with the above mentioned additional constituent substances.

### EXAMPLES

### Example 1

38% by weight of solid matter, final grain size of the minerals in the range of 1 through 100 nanometers, preferably of 1 through 10 nanometers.

Possible advantageous ranges and one range chosen by way of example will be mentioned. This applies to all of the following examples.
10-30=20% by weight of quartz flour, initial size 5 micrometers
5-30=14% by weight of dolomite rock flour, initial size 2 micrometers,
1-10=3% by weight of magnesium powder, about 3 micrometers,
01-5=1% by weight of methyl cellulose,
10-60=38% by weight of vegetable oil (e.g., safflower oil),
2-40=22% by weight of water.

The product is called liquidum Th (with ricinus oil in lieu of the vegetable oil) and has been approved as a dietary supplement. The use of the vegetable oil has the advantage of avoiding the slight laxative effect of the ricinus oil.

### Example 2

Composition of an Oil Emulsion with Minerals and Precipitated Silicic Adic of the Invention:

38% by weight of solid matter, final grain size of the minerals in the range of 1 through 100 nanometers, preferably of 1 through 10 nanometers.
10-30=20% by weight of precipitated silicic acid,
5-30=14% by weight of dolomite rock flour, initial size 2 micrometers,
1-10=3% by weight of magnesium powder, about 3 micrometers,
01-5=1% by weight of methyl cellulose,
10-60=38% by weight of vegetable oil (e.g., safflower oil),
2-40=22% by weight of water.

### Example 3

Composition of a Drug of the Invention in the Form of an Aqueous Solution with Quartz Flour or Precipitated Silicic Acid:

38% by weight of solid matter, final grain size of the minerals in the range of 1 through 100 nanometers, preferably of 1 through 10 nanometers.
10-30=20% by weight of precipitated silicic acid or quartz flour,
5-30=14% by weight of dolomite rock flour, initial size 2 micrometers,
1-10=3% by weight of magnesium powder, about 3 micrometers,
01-5=1% by weight of methyl cellulose,
40-80=60% by weight of water.

### Example 4

Composition of a Drug of the Invention in the Form of an Alcoholic Solution with Quartz Flour or Precipitated Silicic Acid:

38% by weight of solid matter, final grain size of the minerals in the range of 1 through 100 nanometers, preferably of 1 through 10 nanometers.
10-30=20% by weight of precipitated silicic acid or quartz flour,
5-30=14% by weight of dolomite rock flour, initial size 2 micrometers,
1-10=3% by weight of magnesium powder, about 3 micrometers,
01-5=1% by weight of methyl cellulose,
40-80=60% by weight of alcohol.

The mixtures of the examples 1 through 4 are first intensively kneaded in a kneader at a temperature of about 50° C. and then supplied inline to a colloid mill. The colloid mill is a staggered tooth mill with a transfer device and a rotor for cooling. At low speed, the minerals are ground to a size of 10⁻⁹ m. The vegetable oil emulsifies in the warm water and is stabilized by the methyl cellulose. The milling head needs to be cooled during milling to prevent overheating. The thus obtained emulsion is stable and can be diluted further with water and incorporated.

The solutions of the examples 1 through 4 have proved effective against cancer cells whereas coarser ground minerals of 10 μm have shown no effect. The solution moreover proved to only target the cancer cells, not the healthy ones.

### Example 5

Composition of a Powder of the Invention Based on the Liquid Products Described in the Examples 2 and 3:

5-25% by weight of the liquidum of the examples 1 or 2,
60-80% by weight of aluminium silicate, more specifically of natural zeolite having a grain size of 10 to 70, more specifically of 40 μm,
5-25% by weight of dolomite powder having a grain size of 2 to 30, more specifically of 10 μm.

Clinical testing showed that the drug of the invention has the following effects:
improved tolerance to chemotherapy and radiotherapy,
tumor growth suppression,
tumor induration (mineralization),
partial tumor encapsulation and reduction,
improved general condition,
elimination of inflammatory processes as a side effect of radiotherapy and chemotherapy, e.g., of the mucous membranes of the mouth.

Further, first tests showed that the drug of the invention is virostatic. First successful outcomes in the treatment of AIDS (HIV) and SARS have been achieved. In this respect, the applicant reserves the right to file a continuation-in-part application.

I claim:

1-20. (canceled)

21. A drug for the treatment of cancer in a mammal comprising at least one mineral component having a grain size in the range of from about 1 nm to about 150 nm.

22. The drug of claim 21, wherein the grain size is in the range of from about 1 nm to about 20 nm.

23. The drug of claim 21, wherein the mineral component comprises a member selected from the group consisting of quartz, dolomite, magnesium, and combinations thereof.

24. The drug of claim 23, wherein the mineral component further comprises precipitated silicic acid.

25. The drug of claim 24, further comprising an aluminium silicate.

26. The drug of claim 25, wherein said aluminium silicate comprises a natural zeolite having a grain size from about 10 μm to about 70 μm.

27. The drug of claim 26, further comprising a dolomite powder having a grain size from about 2 μm to about 30 μm.

28. The drug of claim 27, wherein the drug comprises about 70% by weight of aluminium silicate, 15% by weight of dolomite powder, and about 15% by weight of at least one other mineral, an excipient, a carrier or a combination thereof.

29. The drug of claim 28, wherein methyl cellulose is added to the mineral component.

30. The drug of claim 27, wherein the drug further comprises from about 30% (w/w) to about 80% (w/w) water.

31. The drug of claim 29, wherein the drug further comprises the addition of an oil-water emulsion.

32. The drug of claim 31, wherein the oil-water emulsion comprises a vegetable oil, a safflower oil or a combination thereof.

33. The drug of claim 31, wherein the drug further comprises the addition of an emulsion of polydimethylsiloxane and water.

34. A method of manufacturing a drug useful for treating cancerous conditions in a mammal comprising the steps of:
providing at least one mineral component; and
forming a mineral powder drug from said mineral component,
wherein the mineral powder has a grain size of from about 1 nm to about 150 nm.

35. The method of claim 34, wherein said drug has a grain size of from about 1 nm to about 20 nm.

36. The method of claim 34, wherein the mineral component comprises a member selected from the group consisting of quartz, dolomite, magnesium or a combination thereof.

37. The method of claim 36, wherein the drug further comprises the addition of precipitated silicic acid.

38. The method of claim 37, wherein the drug further comprises the addition of an aluminium silicate.

39. The method of claim 38, wherein said aluminium silicate comprises a natural zeolite having a grain size from about 10 μm to about 70 μm.

40. The method of claim 39, comprising the addition of a dolomite powder having a grain size from about 2 μm to about 30 μm.

41. The method of claim 40, wherein the drug comprises about 70% by weight of aluminium silicate, 15% by weight of dolomite powder, and about 15% by weight of at least one other mineral, an excipient or a combination thereof.

42. The method of claim 41, wherein the drug further comprises the addition of methyl cellulose.

43. The method of claim 42, wherein the drug further comprises the addition of water.

44. The method of claim 40, wherein the drug further comprises water in a mass fraction of about 60% of the final product.

45. The method of claim 43, wherein the drug further comprises the addition of an oil-water emulsion.

46. The method of claim 45, wherein the oil comprises a vegetable oil, a safflower oil or a combination thereof.

47. The method of claim 45, wherein the drug comprises an emulsion of polydimethylsiloxane and water.
